(19) United States
(12) Patent Application Publication
(10) Pub. No.: US 2006/0228309 A1
(43) Pub. Date: Oct. 12, 2006

(54) Skin lightening composition comprising an extract of plants from the families of symplocos or rubia

(76) Inventors: Indu Mani, Bangalore (IN); Nirmala Nair, Bangalore (IN); Anisha Pargal, Mumbai (IN); Subama Saha, Bangalore (IN); Nagalakshmi Surendra, Bangalore (IN); Shilpa Vora, Bangalore (IN)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 10/559,115

(22) PCT Filed: May 28, 2004
(86) PCT No.: PCT/EP04/05800

§ 371 Date: Jun. 8, 2006

(30) Foreign Application Priority Data
Jun. 3, 2003 (IN) 568/MUM/2003
Sep. 4, 2003 (GB) 0320740.4

(51) Int. Cl. A61K 8/97; A61K 8/27; A61K 36/64

(52) U.S. Cl. 424/59; 424/62; 424/74

(57) ABSTRACT

A cosmetic skin lightening composition, comprising 0.1-50 % by weight of an extract of plants from the families of Symplocos, Rubia or a mixture thereof.

### TECHNICAL FIELD OF THE INVENTION

The present invention relates to an improved cosmetic composition for topical application to human skin to provide enhanced protection from sunlight. The invention particularly relates to a skin lightening composition prepared from natural actives.

### BACKGROUND OF THE PRIOR ART

Melanin is the black pigment synthesised by the action of the enzyme tyrosinase on the amino acid tyrosine. The reaction takes place in organelles called melanosomes contained within cells called melanocytes. Melanocytes transfer melanosomes with melanin to neighbouring keratinocytes which harbour these organelles till they are themselves shed from the body from the superficial layers of the skin. The intensity of the skin colour is directly related to the number, the size, melanin content, the rate of formation and transfer of melanosomes to keratinocytes and degradation of melanin in keratinocytes. Melanin is also an important protectant of skin and tissues beneath the skin as it has the capacity to absorb incident ultraviolet light.

The UV range is divided into three regions, UV-A, having a wavelength of from about 320 to 400 nm which gives a tanning effect without inflammation; UV-B, having a wavelength of from about 290-320 nm which is responsible for erythema (sunburn) and eventually for tanning; and UV-C, having a wavelength of from about 200-290 nm, normally absorbed by the ozone layer in the earth's atmosphere, but which is potentially very damaging to the skin.

Melanogenesis and pigmentation of the skin are closely related responses to irradiation by UV light. Exposure to sunlight leads to darkening of the skin by immediate pigment darkening of already formed melanin, and also by formation of new melanin. Many skin compositions are formulated for the purpose of maintaining the colour of the skin against darkening following exposure to ultra-violet light. These compositions have been based on materials which deflect and scatter incident ultra-violet light of the wavelength which produce burning and tanning of the skin or which absorb this light.

Conventional skin lightening compositions are based on sunscreens or skin lightening agents. The latter are believed to control dispersion of melanosomes or inhibit tyrosinase. Sunscreens alone can not lighten the skin beyond the natural skin colour and their only action is to reduce the ingress of ambient ultraviolet radiation into the skin. Thus, they exert their effect only during the day. Some of the compounds used as skin lightening agents are also known to have undesirable side effects.

Several cosmetic products formulated using plant parts are available in the market. The advantages of plant source of actives in various cosmetic preparations such as skin creams, shampoos, hair applications, perfumes, soothing and nourishing creams etc has been well established. Synthetic actives are considered to be harsher than similar actives obtained from natural sources such as plants.

US20030096023 discloses tyrosinase inhibitors obtained from several dicotyledonous plant species thus leading to skin lightening. The plants are those belonging to the family Polygonaceae, Rosaceae, and Onagraceae.

The present inventors have identified that plant extracts of the species belonging to the genera Symplocos or Rubia on topical application to the skin give improved skin lightening benefits. It is possible to formulate cosmetic compositions incorporating the extracts from these plants to provide protection from the UV damage caused by the sun's radiation and also bring about lightening of the skin.

JP2001192317 (Shiseido) 2001, discloses matrix metalloproteinases inhibitors from several plants including Symplocos. Such an inhibition provides benefit in preventing wrinkles, skin aging.

U.S. Pat. No. 6,258,344 (Procter, 2001), discloses skin lightening compositions having certain hydroquinone derivatives, and lists Rubia under the class of natural anti-inflammatory agents.

JP09175962 (Lion, 1997), discloses use of plants belonging to the family of Rubia cordifolia, in a hair dyeing composition. This refers to darkening of hair which is contrary to the skin lightening effect.

JP2001163755 (Shiseido, 2001), discloses the use of Glycyrrhiza in preparations for external use for skin to obtain skin bleaching effect and JP11021228 (Kanebo, 1999) discloses Glycyrrhiza as an anti-inflammatory agent.

U.S. Pat. No. 6,455,057 (Elizabeth Arden , 2002), discloses a skin care composition comprising petroselinic acid and/or derivatives thereof; a phenolic compound and/or mixtures thereof; and a dermatologically acceptable vehicle for treating wrinkles and soothing sensitive skin. However, they disclose coriander seed oil as a source of petroselinic acid.

However, none of the prior art teaches the skin lightening and protection against damaging effects of ultraviolet radiation by using the extracts of Symplocos or Rubia and cosmetic compositions comprising them.

It is the object of the invention to provide a synergistic cosmetic composition comprising skin lightening actives selected from plant sources.

It is another object of the invention to provide a synergistic cosmetic composition that will provide protection against the damaging effects of ultra violet radiation by the use of actives from natural sources.

It is yet another object of the invention to provide a synergistic cosmetic composition that will provide skin lightening with out requiring the use of chemical actives.

### SUMMARY OF THE INVENTION

According to the present invention there is provided a cosmetic skin lightening composition comprising an extract of the plant Symplocos or Rubia or a mixture thereof.

According to the preferred aspect of the present invention there is provided a cosmetic skin lightening composition comprising 0.1-50% by weight of the composition an extract of the plant Symplocos or Rubia or a mixture thereof.

According to the preferred aspect of the present invention there is provided a cosmetic skin lightening composition comprising:
i. 0.1-50% by weight of the composition an extract of the plant Symplocos or Rubia or a mixture thereof;
ii. 0.1 to 10% by weight of a sunscreen.

The plant parts used in the composition may be selected from any part of the plant but preferably from the stem, bark, leaves, flowers, or roots. It is particularly preferred that the bark of the plant is used.

The extract is prepared using any suitable solvent and particularly-preferred solvent is water.

### DETAILED DESCRIPTION OF THE INVENTION

It is an essential aspect of the present invention that the plant extracts of Symplocos or Rubia are incorporated in the cosmetic composition. However, other plant extracts from Glycyrrhiza, Coriandum, Acorus and useful conventional ingredients may be added to the composition.

Symplocos, is a genus belonging to the family Symplocaceae, commonly available in India. It has several species of which S. recemosa, S. paniculata and S. cochinchinensis are the preferred species for use in the composition.

Rubia, is a genus belonging to the family Rubiaceae of which R. cordifolia, commonly available in India is the preferred species.

The conventional ingredients may be in the nature of sunscreens, other skin lightening agents chosen from natural or other synthetic sources, moisturizers, humectants, benefit agents, perfumes etc.

The composition of the invention may optionally comprise from 0.1 to 10%, preferably 0.5 to 5% by weight of one or more skin whitening agents. The skin whitening agent is preferably chosen from niacin, niacinamide or a precursor thereof that is capable of releasing niacinamide on the skin. Niacinamide is the amide of niacin and is also known as nicotinamide or pyridine-3-carboxylic acid. An example of a compound which is a niacinamide precursor is niacinamide ascorbate. Other suitable skin whitening agents include extracts of placenta, hydroquinone and derivatives (eg. arbutin), kojic acid, dicarboxylic acids (azelaic acid, sebacic acid), represented by the formula HOOC—(CₓH_{y})—COOH where x=4 to 20 and y=6 to 40, ascorbic acid and derivatives thereof, hydroxy acids (lactic acid, glycolic acid, malic acid, tartaric acid etc), ferulic acid, retinol and derivatives or any other known skin whitening compounds.

The sunscreens used may be organic or inorganic in nature and are preferably chosen from 4-tertiary butyl-4′-methoxy dibenzoylmethane, available under the trade name PARSOL 1789 ex Givaudan, 2-ethyl hexyl methoxy cinnamate, available under the trade name PARSOL MCX ex Givaudan or mixtures of the two sunscreen compounds. The composition comprises 0.1 to 10% by weight of the composition and preferably 0.1 to 5% by weight of the composition of a sunscreen compound. Inorganic sunscreens are for e.g. titanium dioxide, zinc oxide, and preferably in the micronized form.

The vehicle which forms part of the cosmetic composition is one or more substances which are mutually compatible with the sunscreen and if present, the skin whitening agent, and does not harm the skin. The vehicle can act as a diluent, dispersant or carrier for the other ingredients of the composition, and is therefore intended to ensure that they can be readily applied to and distributed evenly over the skin at an appropriate concentration.

The vehicles that can be used in compositions according to the invention can include water, powder absorbents, binders and carriers and liquids such as emollients, propellants, solvents, humectants and thickeners.

Examples of moisturisers and humectants include polyols, glycerol, cetyl alcohol, carbopol 934, ethoxylated castor oil, paraffin oils, lanolin and its derivatives. Silicone compounds such as silicone surfactants like DC3225C (Dow Corning) and/or silicone emollients, silicone oil (DC-200 Ex-Dow Corning) may also be included.

Compositions according to the invention can be prepared for topical application to the skin in the form of conventional product types such as creams, lotions, ointments and aerosol products.

The invention will now be illustrated by way of Examples. The Examples are for illustration only and do not in any way restrict the scope of the invention.

### EXAMPLES

In order to test the efficacy of an active in vitro three different cell based assays were used. The herb extract was used as the active that was prepared by the following procedure.

Herbs Tested:

The barks of Rubia cordifolia (A), and Symplocos racemosa (B), were used in the different tests mentioned below.

Extraction Procedure of Herbs:

A 12.5% weight/Volume extract of the herbs was prepared in water. This extract was centrifuged at 14,000×g for 10 min, filtered through a 0.2 micron filter, and 20 micro liter of stock/ml of cell culture media (=0.25% w/v) was used for all the cell culture assays.

i. In vitro Synthesis of Melanin:

The effect of the active on melanin synthesis was tested using melanocytes in culture. B16 melanocytes were grown in culture till they were about 60-70% confluent. The active was then added to the media and the cells were grown till they were confluent. The cells were then harvested and their melanin content was measured at 400 nm using a spectrophotometer. The effect of the actives are presented as a percent of the melanin content of control cells (where the active was not provided) in Table 1.

TABLE 1

| | Melanin Content (% of control) |
|---|---|
| Treatment | Mean ± SEM |
| A | 131.5 |
| B | 89.1 |
| A + B | 95.1 |

The data show that Rubia does not have an influence on the amount of melanin formed while Symplocos reduces the amount of melanin formed. A combination of Rubia and Symplocos show a synergistic reduction in the amount of melanin formed.

ii. Melanin Uptake by Keratinocytes:

The effects of the active on melanin uptake by keratinocytes were tested using HaCaT keratinocytes in culture. HaCaT keratinocytes were cultured till they reached confluence. Cells were then switched to media with no serum and 25 micro gram of synthetic melanin was added to the cells along with the actives. After an overnight incubation, cells were washed and melanin content was measured using a spectrophotometer. The effect of the actives is presented as a percent of the melanin content of control cells in Table 2.

TABLE 2

| | Melanin uptake (% of control) |
|---|---|
| Treatment | Mean ± SEM |
| A | 94.6 |
| B | 89.78 |
| A + B | 89.6 |

The data show that both Rubia and Symplocos alone and in combination decreased the melanin uptake.

iii. Effect on Skin Lightening on a Human Panel:

Combinations of herbs comprising Rubia cordifolia and Symplocos racemosa were formulated into cosmetic creams whose formulation is presented in table 3 and tested on human volunteers in a short-term skin lightening clinical. Untreated and Placebo cream without the herbal extract were maintained for comparison and the results on skin lightening are presented in table 4.

Specific sites were marked on the forearms of 15 volunteers for cream application. The initial skin colour reading on each site served as untreated control. Study personnel applied 3 mg of cream on to each test site. This process was repeated for a period of 10 days. On the 11^{th} day, the subjects were clinically evaluated and scored on a colour scale of 1 to 10 where 1 is the lightest and 10 is the darkest. The data presented in table 4 is the difference in skin colour before and after treatment.

TABLE 3

| Ingredients | % w/w |
|---|---|
| Glycerin | 1.0 |
| Potassium Hydroxide | 0.57 |
| Herbal extract | 30.0 |
| Stearic acid | 10.0 |
| Cetyl Alcohol | 0.6 |
| Preservatives | 0.3 |
| Silicon Oil | 0.5 |
| Water | To 100 |

TABLE 4

| Treatment | Skin lightening |
|---|---|
| Untreated | 0 |
| Placebo cream | 0.29 |
| Herbal cream | 0.39 |

The data show that the herbal extracts even when formulated into a cosmetic composition showed significant skin lightening benefits.

Thus it is apparent from the above that it has been possible to formulate cosmetic compositions using extracts from plants to provide significant skin lightening benefits.

1. A cosmetic skin lightening composition, comprising 0.1 -50% by weight of extracts of plants from the genera Symplocos and Rubia; the extract of Symplocos being selected from Symplocos recemosa, Symplocos paniculata, Symplocos cochinchinensis or mixtures thereof; the extract of Rubia being Rubia cordifolia.

2. The cosmetic skin lightening composition of claim 1, additionally comprising 0.1-10% by weight of a sunscreen.

3. The cosmetic skin lightening composition of claim 1, further comprising 0.1-10% by weight of one or more skin whitening agents.

4. The cosmetic skin lightening composition of claim 1, wherein the extract of Symplocos is Symplocos recemosa.

5. The cosmetic skin lightening composition of claim 1, wherein the sunscreen is selected from 4-tertiary butyl-4′-methyoxy dibenzoylmethane, 2-ethyl hexyl methoxy cinnamate, micronized titanium dioxide, or micronized zinc oxide.

6. The cosmetic skin lightening composition of claim 3, wherein the skin whitening agent is niacin, niacinamide or a pre thereof.

7. The cosmetic skin lightening composition of claim 1 further comprising extracts of Glycyrrhiza, Coriandum, Acorus or mixture thereof.

8. The cosmetic skin lightening composition of claim 1 wherein the Symplocos or Rubia is extracted from the bark of the plant.
